(19) United States
(12) Patent Application Publication
(10) Pub. No.: US 2019/0358411 A1
(43) Pub. Date: Nov. 28, 2019

(54) INJECTION DEVICES AND RELATED METHODS OF USE AND ASSEMBLY

(71) Applicant: AMGEN INC., Thousand Oaks, CA (US)

(72) Inventor: Lawrence S. Ring, Laguna Beach, CA (US)

(21) Appl. No.: 16/476,694

(22) PCT Filed: Jan. 16, 2018
(86) PCT No.: PCT/US18/13815

§ 371 Date: Jul. 9, 2019

Related U.S. Application Data
(60) Provisional application No. 62/447,174, filed on Jan. 17, 2017.

(51) Int. Cl. A61M 5/32; A61M 5/20

(52) U.S. Cl. CPC A61M 5/3287; A61M 2005/1585; A61M 5/326; A61M 5/2033

(57) ABSTRACT

Injection devices for drug delivery and related methods of use and manufacture are disclosed. The injection device may include an outer casing, a container disposed in the outer casing, an injection drive mechanism, and an instructional marker disposed on an exterior surface of the outer casing. The container may include an interior chamber for storing a drug, a subcutaneous delivery member, and a stopper movably disposed in the interior chamber. The injection drive mechanism may be configured to move the stopper to expel the drug through an opening in a distal end of the subcutaneous delivery member upon activation. The instructional marker may indicate a tiling direction for tilting the injection device when the subcutaneous delivery member is inserted into a patient so as to inhibit tissue from occluding the opening in the distal end of the subcutaneous delivery member.

### CROSS-REFERENCE TO RELATED APPLICATION

The priority benefit of U.S. Provisional Patent Application No. 62/447,174, filed Jan. 17, 2017, is claimed, and the entire contents thereof are expressly incorporated herein by reference.

### FIELD OF DISCLOSURE

The present disclosure generally relates to injection devices and related methods. More particularly, the present disclosure is directed to injection devices and methods for automatically or semi-automatically delivering a drug subcutaneously to a patient via a delivery member such as a needle or cannula.

### BACKGROUND

Automated injection devices for drug delivery, including autoinjectors and on-body injectors, offer several benefits over traditional methods of drug delivery using, for example, conventional syringes. One of these benefits is their simplicity and ease of use, which makes it possible for a patient to self-administer a drug with little or no assistance from a medical professional.

Many injection devices use a coil or other spring structure to provide energy for driving a plunger to expel the drug from a primary container. Springs typically exhibit an inverse relationship between force and displacement. Consequently, the force provided by a spring actuator of an injection device at the end of a plunger stroke may be substantially less than at the beginning of the stroke. In some cases, this can prevent proper dose completion. High viscosity drugs such as biologics tend to exacerbate this problem because of their resistance to being pushed through the small passageway of a needle or cannula.

Loading the injection device with a more powerful spring (i.e., a spring with a higher spring constant) can reduce the likelihood of incomplete dosing, but this approach may have undesirable consequences. Kinetic energy is proportional to velocity squared; therefore, an incremental increase to the spring constant can result in a large change to the kinetic energy applied to the drug and/or primary container. The patient may feel or hear this excessive energy in the form of a slap or similar physical bump as the spring driven plunger impacts the stopper of the primary container. Such mechanical bumps can be distracting or even disturbing to the patient. There is even a chance that the slap or bump generated by the excessive energy may have severe consequences, such as breakage of the primary container and/or damage to the drug product itself by way of shear loading.

The present disclosure sets forth injection devices and methods embodying advantageous alternatives to existing injection devices and methods, and that may address one or more of the challenges or needs mentioned herein, as well as provide other benefits and advantages.

### SUMMARY

One aspect of the present disclosure provides an injection device for drug delivery including an outer casing, a container disposed in the outer casing, an injection drive mechanism, and an instructional marker. The container may include an interior chamber for storing a drug, a subcutaneous delivery member, and a stopper movably disposed in the interior chamber. The injection drive mechanism may be configured to move the stopper to expel the drug through an opening in a distal end of the subcutaneous delivery member upon activation. The instructional marker may indicate a tiling direction for tilting the injection device when the subcutaneous delivery member is inserted into a patient so as to inhibit tissue from occluding the opening in the distal end of the subcutaneous delivery member.

Another aspect of the present disclosure provides a method of assembling an injection device for drug delivery. The method may include: (a) disposing a container within an outer casing, wherein the container includes an interior chamber for storing a drug, a subcutaneous delivery member, and a stopper movably disposed in the interior chamber to expel the drug through an opening defined by a bevel formed in a distal end of the delivery member; (b) rotationally aligning the bevel of the subcutaneous delivery member and a target portion of the outer casing; (c) marking an exterior surface of the outer casing with an instructional marker, wherein the instructional marker indicates a tilting direction for tilting the injection device when the subcutaneous delivery member is inserted into a patient so as to inhibit tissue from occluding the opening in the distal end of the subcutaneous delivery member.

Yet another aspect of the present disclosure provides a method of using an injection device for drug delivery. The method may include: (a) providing an injection device including an outer casing, a container disposed in the outer casing and including an interior chamber for storing the drug, a subcutaneous delivery member, a stopper movably disposed in the interior chamber, and an injection drive mechanism configured to move the stopper to expel the drug through an opening in a distal end of the subcutaneous delivery member upon activation; (b) inserting the distal end of the subcutaneous delivery member into the patient with the subcutaneous delivery member having a first orientation relative to an injection site of the patient; (c) tilting the injection device in a first direction such that the subcutaneous delivery member has a second orientation relative to the injection site of the patient; and (d) activating the injection drive mechanism to subcutaneously deliver the drug to the patient while maintaining the subcutaneous delivery member in the second orientation.

### BRIEF DESCRIPTION OF THE DRAWINGS

It is believed that the disclosure will be more fully understood from the following description taken in conjunction with the accompanying drawings. Some of the drawings may have been simplified by the omission of selected elements for the purpose of more clearly showing other elements. Such omissions of elements in some drawings are not necessarily indicative of the presence or absence of particular elements in any of the exemplary embodiments, except as may be explicitly delineated in the corresponding written description. Also, none of the drawings is necessarily to scale.

FIG. 1 illustrates a perspective view of an embodiment of an injection device in accordance with principles of the present disclosure.

FIG. 2 illustrates a cross-sectional view of the injection device depicted in FIG. 1.

FIGS. 3A-3G illustrate a sequence of steps for using the injection device of FIG. 1 to deliver a drug subcutaneously to a patient, in accordance with one embodiment of the present disclosure.

FIG. 4A is an enlarged perspective view of a distal end of a subcutaneous delivery member of the injection device shown in FIG. 1.

FIG. 4B is an enlarged side view of the distal end of the subcutaneous delivery member of the injection device illustrated in FIG. 1.

FIG. 5 is a side view of another embodiment of a distal end of a subcutaneous delivery member in accordance with principles of the present disclosure.

### DETAILED DESCRIPTION

In general, the present disclosure provides devices and methods for reducing the injection resistance (e.g., backpressure) experienced by a needle or other delivery member during subcutaneous delivery of a drug. Subcutaneous delivery generally involves piercing a patient's skin with a distal end of the delivery member and subsequently expelling the drug through an opening in the distal end of the delivery member. In some instances, the patient's tissue may partially, or completely, occlude the opening in the delivery member following insertion of the delivery member. Consequently, the patient's tissue may resist the flow of the drug through the opening, thereby causing injection resistance which the drive mechanism of the injection device must overcome to complete delivery of a dose of the drug.

It has been found by the present inventor that the amount of injection resistance is correlated to the orientation of the delivery member during drug delivery. More particularly, it has been found that injection resistance is reduced by tilting the delivery member relative to the patient. For example, after inserting the delivery member into the patient in a perpendicular orientation relative to a reference point, injection resistance may be reduced by tilting the delivery member so that it has a non-perpendicular orientation relative to said reference point. This approach is particularly effective at reducing injection resistance if the delivery member is tilted in a direction away from a lateral side of the delivery member having the opening(s) for expelling the drug. For example, where the distal end of the delivery member has a bevel with an opening extending therethrough, tilting the delivery member in a direction away from a lateral side of the delivery member including the bevel following insertion may inhibit or prevent the patient's tissue from occluding (e.g., obstructing) the opening in the drug delivery member.

Lower injection resistance reduces the risk of incomplete dose delivery, which is particularly beneficial in the case of high viscosity drugs such as biologics. Furthermore, with less injection resistance, it may be possible to use a less powerful drive mechanism to expel the drug from the device. In addition, the slap or similar physical bump mentioned above, which is oftentimes experienced when the drive mechanism initially releases an excessive amount of energy, may be avoided or at least diminished by the disclosed tilting procedure. Moreover, breakage of the drug storage container and/or damage to the drug itself due to excessive shear load is less likely.

Many injection devices possess a needle or other delivery member that is hidden from view prior to use. As such, a user (e.g., a patient, a caregiver, a healthcare provider, etc.) may be unable to identify the side of the delivery member having the opening and/or bevel. Thus, it may be difficult, or even impossible, for the user to determine the direction in which to tilt the delivery member to reduce injection resistance. The presently disclosed injection devices and methods advantageously help the user determine the appropriate direction for tilting the injection device, as well as the optimal amount of tilting, in an effort to reduce injection resistance.

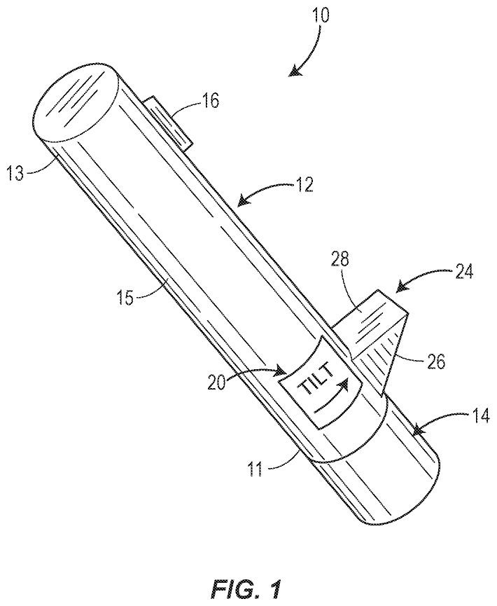

FIG. 1 is a perspective view of one embodiment of an injection device 10 according to the present disclosure. The injection device 10 may be configured as a single-use, disposable injector or a multiple-use reusable injector. The injection device 10 may be configured to deliver any suitable medicament or drug including those having a high viscosity such as a biologic drug. Further, the injection device 10 may be configured as an autoinjector for self-administration, although the injection device 10 can also be used by a caregiver or a formally trained healthcare provider to administer an injection. In embodiments where the injection device 10 is configured as an autoinjector, the injection device 10 may be held in the users hand over the duration of drug delivery. In other embodiments, where drug delivery may be delayed or take several minutes or hours, the injection device 10 may be configured as an on-body injector which is releasably attached to the patient's skin via, for example, an adhesive.

Referring still to FIG. 1, the injection device 10 may include an elongated, housing or outer casing 12. The outer casing 12 may be a single, unitary component or a multiple component assembly. A cap 14 may be removably attached to a distal end 11 of the outer casing 12. Prior to use, the user may detach the cap 14 from the outer casing 12 to expose an opening providing access to a subcutaneous delivery member 42. The injection device 10 may also include a trigger or button member 16 that protrudes outwardly from a proximal end 13 of the outer casing 12. The user may manually depress the button member 16 to activate the injection drive mechanism, as described below in more detail.

Still referring to FIG. 1, an instructional marker 20 may be disposed on the exterior surface 15 of the outer casing 12. In general, the instructional marker 20 may indicate a direction for tilting the injection device 10 in order to inhibit or prevent the patient's tissue from occluding one or more openings in the subcutaneous delivery member 42 during drug delivery. In addition to indicating the tilting direction, the instructional marker 20 may indicate the amount of tilting (e.g., the number of degrees) that is optimal for reducing injection resistance. The instructional marker 20 may be composed of text, graphics, symbols, lines, pictures, or any suitable combination thereof, and/or any other marker that conveys meaning to a user, alone, or in combination with a tilting assist member 24, as depicted. In the illustrated embodiment, a portion of the instructional marker 20 includes both an arrow pointing in the tilting direction and text instructing the user to “TILT” the injection device 10 in the direction of the arrow. This portion of the instructional marker 20 may be applied to the outer casing 12 in any suitable manner, including being printed directly on the exterior surface 15 of the outer casing 12 or being formed as a decal adhered to the exterior surface 15 of the outer casing 12. Furthermore, in some embodiments, the instructional marker 20 may include an electronic display, such as an LED display panel or individual lights, in response to activation of the injection device 10. In yet other embodiments, the instructional marker 20 could include a fluid chamber housing a bubble (or a simulation of a fluid chamber housing a bubble). So configured, the user may be instructed to tilt the injection device 10 until the bubble (or simulated bubble) is aligned between a pair of target lines disposed on the fluid chamber (or simulated fluid chamber).

Still referring to FIG. 1, in some versions, the instructional marker 20 also includes a tilting assist member 24 that may protrude radially outwardly from a distal end 11 of the outer casing 12. The tilting assist member 24 may provide a structure for aiding the user in tilting the injection device 10 in the proper direction and/or to an appropriate angle. In some versions, the tilting assist member 24 may be constructed of the same material or a similar material as the outer casing 12 and may be generally rigidly fixed in the position illustrated in FIG. 1, for example. In other versions, the tilting assist member 24 could be coupled to the outer casing 12 via a hinge that allows the tilting assist member 24 to be folded back onto the outer casing 12, which may facilitate more compact packaging and/or storage. In yet other versions, the tilting assist member 24 my include a more flexible structure such as for example a plastic or cardboard fin or accordion configuration, which could be folded or compressed against and/or partially around the outer casing 12 to facilitate compact storage and/or packaging. In these non-rigid or hinged versions, the user of the injection device 10 would have to properly position the tilting assist member 24 by folding, pivoting, pulling or otherwise, relative to the outer casing 12, prior to tilting the injection device 10 during drug delivery. In yet other forms, the tilting assist member 24 could be constructed of a resilient material such as a foam or a foam rubber, for example. In such a version, the foam material may provide the user with sufficient feedback to indicate proper tilting. Moreover, a resilient version of the tilting assist member 24 could be initially stored in a compressed state against the outer casing 12 with a piece of tape or a label for compact storage and/or packaging. Thus, prior to use, the user would simply peel off the label or tape and the tiling assist member 24 would spring into an active position, shaped as depicted in FIG. 1 or otherwise, for example. Of course, other versions and configurations are possible.

As shown in FIG. 1, the tilting assist member 24 may generally take the shape of a ramp which slopes downwards towards the distal end 11 of the outer casing 12. The inclined surface of the ramp may define an injection site engaging surface 26. To achieve the proper angle for injection, the injection device 10 may be tilted until the injection site engaging surface 26 contacts and/or is flush with the patient's skin 90 at the injection site 92. A finger grip surface 28 may be located proximally of the injection site engaging surface 26 and may, in some injection procedures, be pressed upon by the user to help tilt the injection device 10. A more detailed description of the orientation and use of the surfaces 26 and 28 of the tilting assist member 24 is provided below.

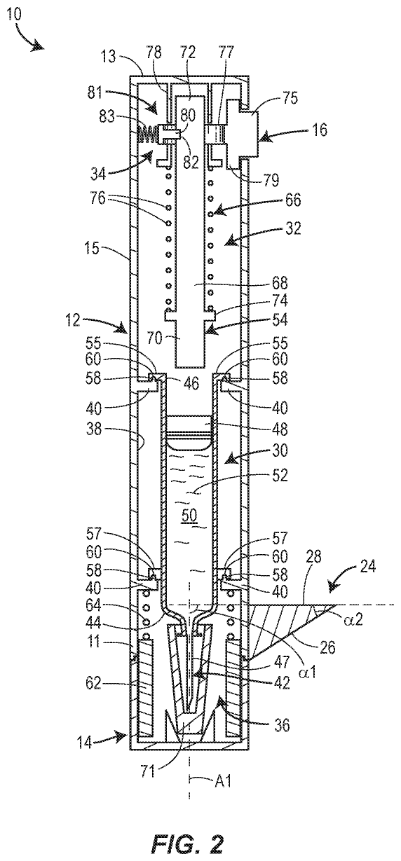

Turning to FIG. 2, the injection device 10 may include one or more of a drug storage container 30, an injection drive mechanism 32, a drive triggering mechanism 34, a guard mechanism 36, each of which may be enclosed within the outer casing 12. In some embodiments, the drug storage container 30 may comprise a conventional glass or plastic syringe or cartridge.

The outer casing 12 may have an interior surface 38 that can include one or more support members 40 fixedly disposed thereon for holding the drug storage container 30 in a fixed manner relative to the outer casing 12. In some embodiments, one or more of the support members 40 may comprise a continuous, annular ledge or shelf. In other embodiments, one or more of the support members 40 may be formed as two or more coplanar ledges or shelf segments.

Furthermore, in some embodiments, one or more of the support members 40 may be configured as a carrier for the drug storage container 30. The carrier may be configured and adapted to move axially and/or allow the drug storage container 30 to move relative to the outer casing 12 to insert a subcutaneous delivery member 42 associated with the drug storage container 30 into the body of the patient after the injection device 10 has been appropriately positioned on the body at a selected injection site.

Referring still to FIG. 2, the drug storage container 30 may include a distal end 44 carrying the subcutaneous delivery member 42 and a proximal end 46 carrying a stopper 48. The subcutaneous delivery member 42 may be a rigid injection needle, a flexible cannula, or any other fluid dispensing element suitable for injecting a drug into the body of the patient. An interior chamber 50 may extend between the distal and proximal ends 44 and 46 of the drug storage container 30, and may be configured to store a drug 52. In some embodiments, the interior chamber 50 of the drug storage container 30 may be pre-filled with the one or more doses of the drug 52. The stopper 48 may be disposed in the interior chamber 50 so that it is axially moveable in a distal direction relative to the remainder of the drug storage container 30 to expel the drug 52 through the subcutaneous delivery member 42. The stopper 48 may sealingly engage the wall defining the interior chamber 50 so that the drug is prevented or inhibited from leaking past the stopper 48. The proximal end 46 of the drug storage container 30 may be open to allow a plunger 54 of the injection mechanism 32 to extend into the drug storage container 30 and push the stopper 48 in the distal direction.

The subcutaneous delivery member 42 may extend linearly along a longitudinal axis A1 between a proximal end 47 and a distal end 49 of the subcutaneous delivery member 42. The proximal end 47 of the subcutaneous delivery member 42 may be connected at the distal end 44 of the drug storage container 30 and in fluid communication with the interior chamber 50. In some embodiments, a rigid connection may be formed between the subcutaneous delivery member 42 and a remainder of the drug storage container 30 such that the subcutaneous delivery member 42 is prevented from rotating and/or translating axially relative to the remainder of the drug storage container 30. In other embodiments, the subcutaneous delivery member 42 may be allowed to move axially relative to the remainder of the drug storage container 30 but not rotate relative to the remainder of the drug storage container 30. The subcutaneous delivery member 42 may include a hollow interior passageway extending between the proximal and distal ends 47 and 49 to allow the drug 52 to flow through the subcutaneous delivery member 42 upon activation of the drive mechanism 32. An opening 53, or multiple openings, may be formed in the distal end 49 of the subcutaneous delivery member 42 to permit the drug 52 expelled from the interior chamber 50 to be delivered subcutaneously to the patient.

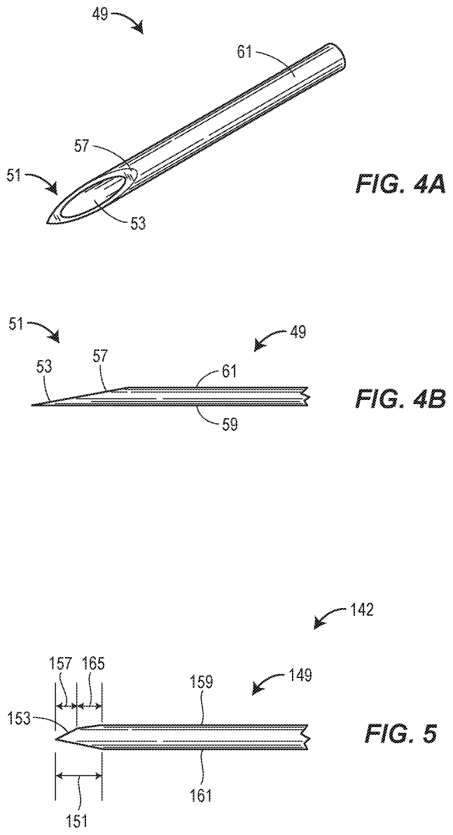

Turning briefly to FIGS. 4A and 4B, in some embodiments the distal end 49 of the subcutaneous delivery member 42 may include a tapered region 51, where the width of the subcutaneous delivery member 42 gradually decreases to a tip which is sharp enough to pierce the patient's skin. In the embodiment illustrated in FIGS. 4A and 4B, the tapered region 51 is defined by a bevel 57 formed in a first lateral side 59 of the distal end 49 of the subcutaneous delivery member 42. Accordingly, the bevel 57 is defined by a plane or cut that intersects the first lateral side 59 of the subcutaneous delivery member 42 at a first axial location along the longitudinal axis A and intersects a second lateral side 61 of the subcutaneous delivery member 42 at a second axial location, where the first axial location is distal to the second axial location along the longitudinal axis A. As described below in more detail, injection resistance may be reduced by tilting the subcutaneous delivery member 42 in a direction away from the second lateral side 61 of the subcutaneous delivery member 42 (i.e., the side of the subcutaneous delivery member 42 including the opening 53 and the bevel 57).

In the embodiment shown in FIGS. 4A and 4B, the opening 53 is included in the tapered region 51 and extends through the bevel 57. In other embodiments, the subcutaneous delivery member 42 may alternatively or additionally include an opening located proximal to the tapered region 51. Furthermore, while the bevel 57 illustrated in FIGS. 4A and 4B is defined by a constant angle relative to the longitudinal axis A1, in other embodiments, the bevel 57 may have multiple sections each defined by a different angle relative to the longitudinal axis A1.

FIG. 5 illustrates an alternative embodiment of a distal end 149 of a subcutaneous delivery member 142. Elements of the subcutaneous delivery member 142 which are similar to the subcutaneous delivery member 42 are designated by the same reference numeral, incremented by 100. A description of many of these elements is abbreviated or even eliminated in the interest of brevity. The tapered region 151 of the subcutaneous delivery member 142 includes both a bevel 157 and a conical portion 165, with the conical portion 165 being disposed proximally of the bevel 157. This configuration may facilitate piercing of the patient's skin or tissue in certain situations.

Configurations of the subcutaneous delivery member are not limited to those described in connection with FIGS. 4A, 4B, and 5. In other embodiments, the subcutaneous delivery member may be configured in the same or a similar manner as those described in U.S. Patent Application Publication No. 2015/0290390, which is hereby incorporated by reference in its entirety for all purposes.

Referring back to FIG. 2, the proximal end 46 of the drug storage container 30 may include one or more flanges 55 which protrude radially outwardly from an outer surface of the drug storage container 30. Additionally or alternatively, the distal end 44 of the drug storage container 30 may include one or more radially outwardly protruding flanges 57. In some embodiments, the flanges 55 and/or 57 may be configured to engage respective ones of the support members 40 protruding from the interior surface 38 of the outer casing 12. In embodiments where the support members 40 are fixed relative to the outer casing 12, the support members 40 may prevent distal advancement of the drug storage container 30 by virtue of their engagement with respective ones of the flanges 55 and/or 57. Furthermore, in some embodiments, the flanges 55 and/or 57 may be omitted.

In some embodiments, the support members 40 may be configured to rotationally fix or lock the drug storage container 30 relative to the outer casing 12 so that the subcutaneous delivery member 42 is also rotationally fixed or locked relative to the outer casing 12. Accordingly, once installed in a particular rotational position relative to the outer casing 12, the subcutaneous delivery member 42 may be inhibited or prevented from rotating out of that rotational position due to, for example, vibrations and/or sudden movements experienced by the injection device 10 during storage and/or transport. In some embodiments, the rotational fixing may be accomplished by one or more axially-extending engagement structures 58 which protrude from the support members 40 as shown in FIG. 2. The engagement structures 58 may be received in, or otherwise cooperate with, corresponding holes or recesses 60 formed in the flanges 55 protruding from the drug storage container 30. In other embodiments, the engagement structures 58 may protrude from one or more of the flanges 55, and the corresponding holes or recesses 60 may be formed in one or more of the support members 40. Furthermore, instead of being integrally formed as part of the support members 40 or the flanges 55, in some embodiments the engagement structures 58 may be separate structures such as pins or screws which extend through overlapping holes formed in the support members 40 and the flanges 55. In still further alternative embodiments, the engagement structures 58 may include a clamp configured to grasp or otherwise constrain a portion of the drug storage container 30 (e.g., a flat side of one or more of the flanges 55) and which employs friction on friction to prevent or inhibit rotation of the drug storage container 30 relative to the outer casing 12.

With continued reference to FIG. 2, a removable shield 71 (e.g., a rigid needle shield) may be installed over the subcutaneous delivery member 42 for maintaining a sterile state prior to use of the injection device 10. In embodiments where the support members 40 are fixed relative to the outer casing 12, the subcutaneous delivery member 42 may extend through an opening in the distal end 11 of the outer casing 12.

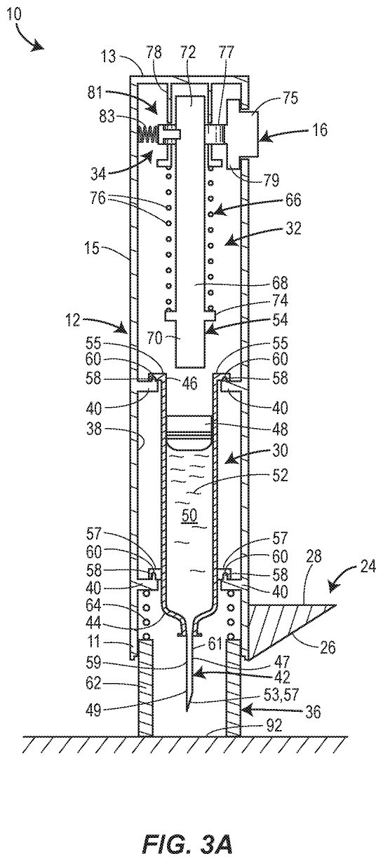

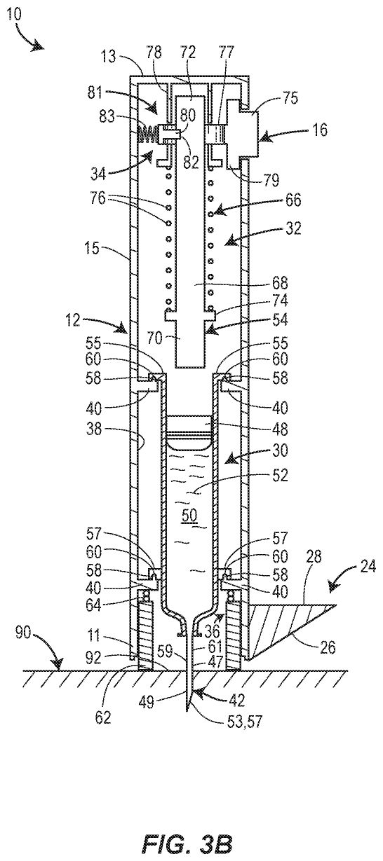

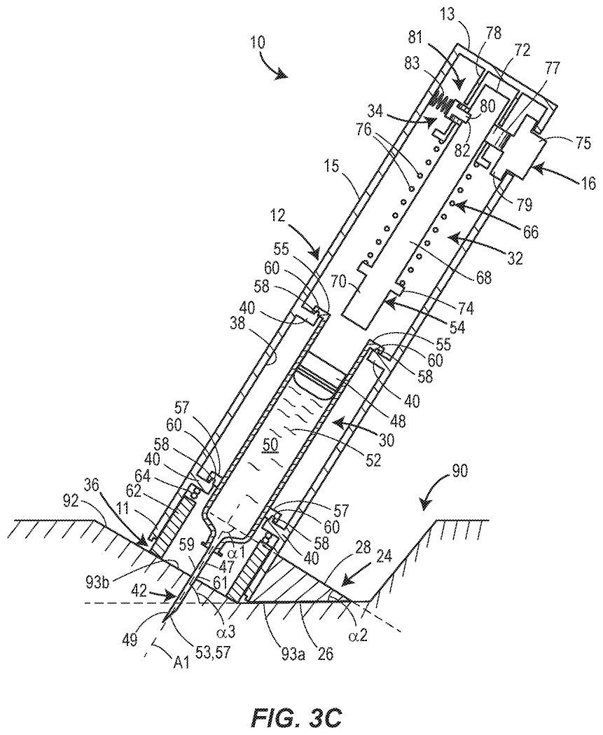

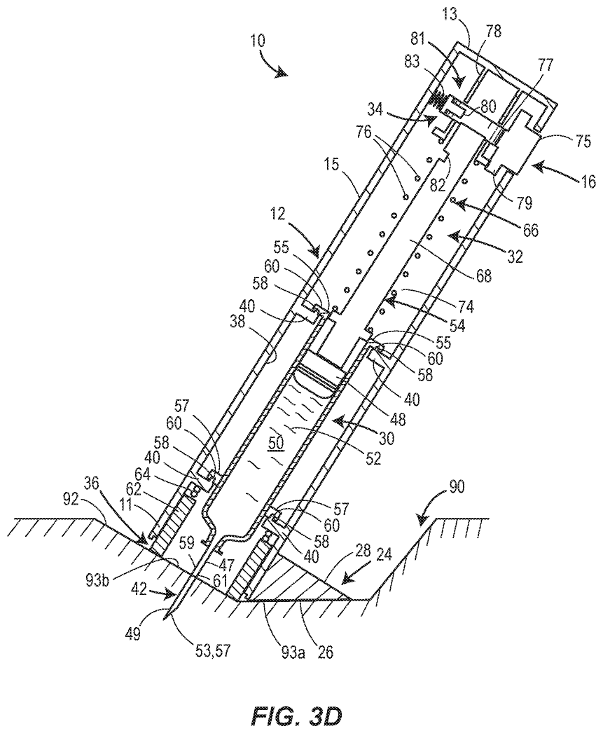

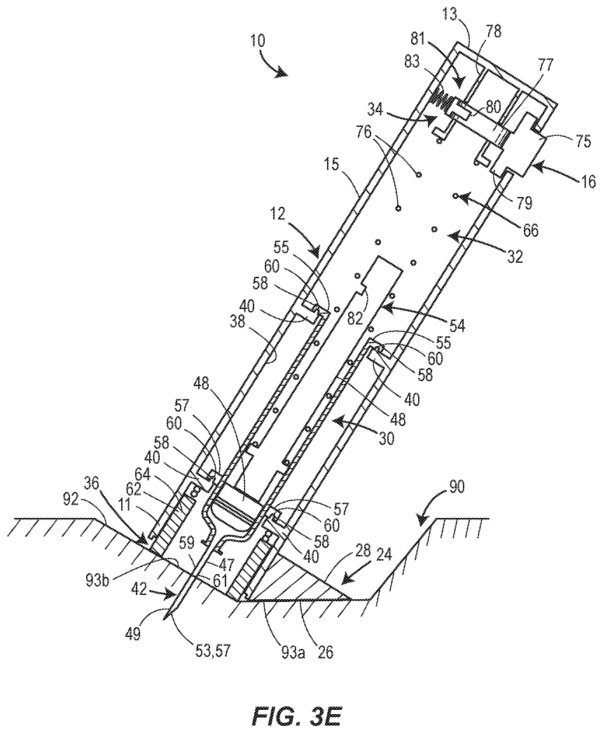

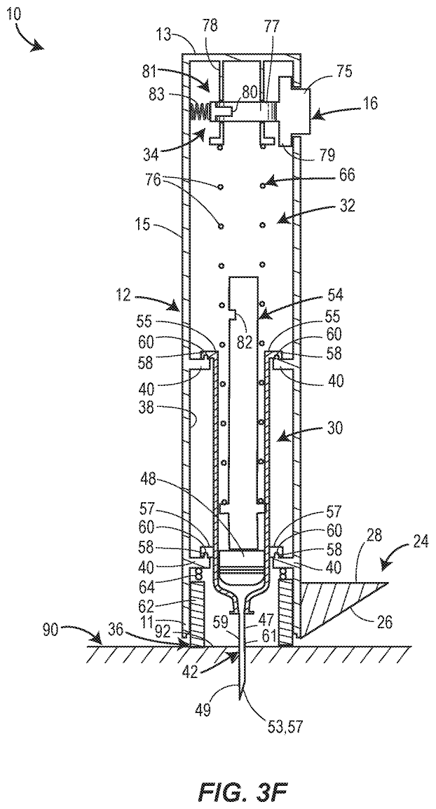

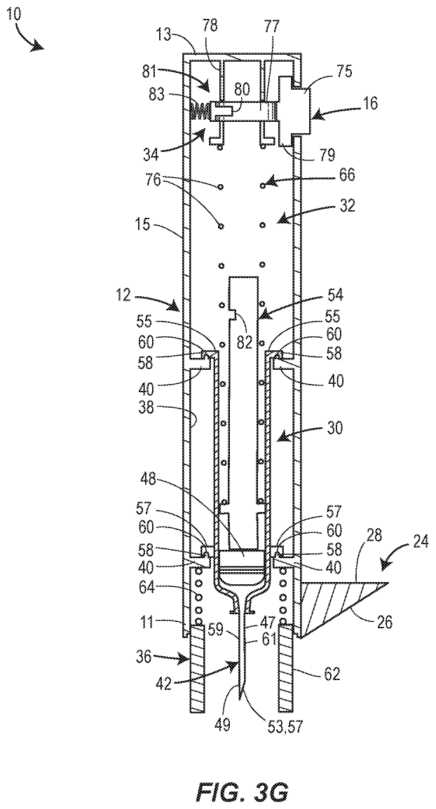

The guard mechanism 36 may prevent the user or patient from contacting or being pierced by the subcutaneous delivery member 42 when the injection device 10 is not being used to administer an injection. As shown in FIG. 2, the guard mechanism 36 may include a guard member 62 movably disposed at the distal end 11 of the outer casing 12. The guard mechanism 36 may further include a biasing member 64 that holds the guard member 62 in an extended position when the injection device 10 is not in use and allows the guard member 62 to retract in the proximal direction relative to the outer casing 12 when the injection device 10 is pressed against the patient's skin 90 at the injection site 90. The guard member 62 remains in an extended position relative to the outer casing 12 via the biasing member 64 when the injection device 10 is not being used to administer an injection, thereby surrounding or covering the subcutaneous delivery member 42. The guard member 62 retracts toward the outer casing 12 when the injection device 10 is pressed against the patient's skin 90 at the injection site 90 to allow the subcutaneous delivery member 42 to penetrate the patient's body (see FIG. 3B). The guard member 62 may have a tubular configuration or any other suitable configuration that is capable of preventing the user or patient from contacting the subcutaneous delivery member 42 when the guard member 62 is in an extended position. The biasing member 64 can include a coil spring or any other suitable mechanism that is capable of holding the guard member 62 in the extended position and allows the guard member 62 to retract toward the outer casing 12 when the injection device 10 is pressed toward the body of the patient at the injection site. The guard mechanism 36 may be configured so that the guard member 62 slides into or over the distal end 11 of the outer casing 12 during retraction of the guard member 62 (see FIG. 3B). The biasing member 64 may be disposed between a proximal end of the guard member 62 and a portion of the outer casing 12 (e.g., one or more of the support members 40 fixedly disposed on the interior surface 38 of the outer casing 12).

Still referring to FIG. 2, the injection drive mechanism 32 may include the plunger 54 and an energy source 66 for driving the plunger 54 into the interior chamber 50 of the drug storage container 30 to expel the drug 52 via the subcutaneous delivery member 42. In embodiments where the drug storage container 30 is adapted to move axially relative to the outer casing 12, the energy source 66 may be configured to drive both the plunger 54 and the drug storage container 30 in the distal axial direction relative to the outer casing 12. The plunger 54 may include a rod member 68 having distal and proximal ends 70 and 72, respectively. The distal end 70 may include an outwardly extending annular flange 74 defining a spring seat.

The energy source 66 may include one or more spring elements. As depicted in FIG. 2, the one or more spring elements may include a coil spring 76. The rod member 68 of the plunger 54 may extend through the coil spring 76 so that one end of the spring 76 engages the annular flange 74. The other end of the spring 76 may engage a tubular protrusion 78 extending axially from the proximal end 13 of the outer casing 12. Prior to operation of the injection device 10, the coil spring 76 may be compressed between the annular flange 74 of the plunger 54 and the tubular protrusion 78, thereby generating a spring biasing force against the annular flange 74 and the tubular protrusion 78. When the injection device 10 is activated by the drive triggering mechanism 34, as explained in further detail below, the coil spring 76 expands in the distal direction, thereby propelling the plunger 54 into the drug storage container 30 to drive the stopper 48 through the interior chamber 44 to expel the drug 52 via the subcutaneous delivery member 42.

In other embodiments, the energy source 66 may alternatively or additionally include a gas pressure or gas releasing assembly. The energy provided by such a gas pressure or gas releasing assembly may operate on the plunger 54 to propel it into the drug storage container 30, thereby driving the stopper 48 through the interior chamber 44 to expel the drug 52 through the subcutaneous delivery member 42.

Still referring to FIG. 2, the drive triggering mechanism 34 may include the button member 16, a plunger release member 81, and a trigger biasing member 83. The button member 16 may allow the drive triggering mechanism 34 to be actuated to administer an injection of the drug 52. The button member 16 may include a head portion 75 surrounded by a peripheral edge portion 79. The head portion 75 may extend above the peripheral edge portion 79 so that it can project through a button aperture formed in the outer casing 12 when the peripheral edge portion 79 contacts the interior surface 38 of the outer casing 12 to allow actuation of the button member 16 by the user. The plunger release member 81 may project from an inner surface of the button member 16. In some embodiments, the plunger release member 81 may include an arm portion 77 having a C-shape that extends partially around the rod member 68 and/or the tubular protrusion 78. A latch or detent member 80 may extend inwardly from the plunger release member 81 and may be received in, or otherwise cooperate with, a recess 82 formed in the outer surface of the rod member 68.

The trigger biasing member 83 may exert a biasing force against the outer edge of the arm portion 77 so that the detent member 80 is securely positioned in the recess 82 of the rod member 68 when the button member 16 is not pressed by the user (i.e., activated). This, in turn, may retain the rod member 68 in the proximal-most axial position relative to the outer casing 12 by preventing the coil spring 76 from expanding, as shown in FIG. 2. This may be referred to as the armed or ready-to-use mode of the injection device 10. The trigger biasing member 83 can comprise a coil spring or any other suitable energy source.

After inserting the subcutaneous delivery member 42 into the body, the user may press the button member 16 into the outer casing 12 against the biasing force of the trigger biasing member 83 to actuate the injection drive mechanism 32 to administer the injection (see FIG. 3D). As a result, the plunger release member 81 moves laterally within the outer casing 12, thereby disengaging the detent member 80. This, in turn, releases the plunger 54 and allows the energy source 66 to propel the plunger 54 into the drug storage container 30 to drive the stopper 48 through the interior chamber 50 to expel the drug 52 through the subcutaneous delivery member 42.

With reference to FIGS. 2 and 3C, the orientation of various surfaces of the tilting assist member 24 and the subcutaneous delivery member 42 will now be described. As shown in FIG. 2, the finger grip surface 28 of the tilting assist member 24 may be arranged relative to the longitudinal axis A1 of the subcutaneous delivery member 42 at a first angle α1; and the injection site engaging surface 26 may be arranged relative to the finger grip surface 28 at a second angle α2. In some embodiments, the first angle α1 may correspond to an angle formed by the intersection between the longitudinal axis A1 of the subcutaneous delivery member 42 and an imaginary plane touching or coincident with the finger grip surface 28 of the tilting assist member 24. In some embodiments, the first angle α1 may be equal to approximately (e.g., ±10%) 90 degrees, or within a range of approximately (e.g., ±10%) 45-90 degrees, or within a range of approximately (e.g., ±10%) 75-90 degrees. In some embodiments, the second angle α2 may less than 90 degrees, or within a range of approximately (e.g., ±10%) 30-120 degrees, or within a range of approximately (e.g., ±10%) 25-65 degrees. It is noted that although the finger grip surface 28 is described as having a first angle α1, this does not necessarily mean that the entire finger grip surface 28 is arranged at the first angle α1. Rather, it simply means that at least a portion of the finger grip surface 28 is arranged at the first angle α1 relative to the longitudinal axis A1 of the subcutaneous delivery member 42. The same applies the injection site engaging surface 26 and its second angle α2 relative to the finger grip surface 28.

Referring to FIG. 3C, at least when the injection device 10 is tilted such that the injection site engaging surface 26 of the tilting assist member 24 contacts and/or is flush with the patient's skin 90 at a peripheral portion 93a of the injection site 92, the longitudinal axis A1 of the subcutaneous delivery member 42 may be arranged relative to the injection site engaging surface 26 of the tilting assist member 24 at a third angle α3. The third angle α3 may correspond to an optimal angle for preventing or inhibiting tissue from occluding the opening 53 in the distal end 44 of the subcutaneous delivery member 42. In some embodiments, the third angle α3 may correspond to an angle formed by the intersection between the longitudinal axis A1 of the subcutaneous delivery member 42 and an imaginary plane touching or coincident with the injection site engaging surface 26 of the tilting assist member 24. In some embodiments, the third angle α3 may less than 90 degrees, or less than or equal to approximately (e.g., ±10%) 85 degrees, or less than or equal to approximately (e.g., ±10%) 80 degrees, or less than or equal to approximately (e.g., ±10%) 75 degrees, or less than or equal to approximately (e.g., ±10%) 70 degrees, or less than or equal to approximately (e.g., ±10%) 65 degrees, or less than or equal to approximately (e.g., ±10%) 60 degrees, or within a range of approximately (e.g., ±10%) 5-85 degrees, or within a range of approximately (e.g., ±10%) 35-85 degrees, or within a range of approximately (e.g., ±10%) 45-85 degrees, or within a range of approximately (e.g., ±10%) 55-85 degrees, or within a range of approximately (e.g., ±10%) 60-85 degrees, or within a range of approximately (e.g., ±10%) 5-80 degrees, or within a range of approximately (e.g., ±10%) 35-80 degrees, or within a range of approximately (e.g., ±10%) 45-80 degrees, or within a range of approximately (e.g., ±10%) 55-80 degrees, or within a range of approximately (e.g., ±10%) 60-80 degrees, or within a range of approximately (e.g., ±10%) 40-75 degrees, or within a range of approximately (e.g., ±10%) 45-75 degrees, or within a range of approximately (e.g., ±10%) 50-75 degrees. Furthermore, in some embodiments, a sum of the first angle α1, the second angle α2, and the third angle α3 may be equal to or substantially equal to 180 degrees.

In embodiments where the longitudinal axis A1 of the subcutaneous delivery member 42 is parallel or substantially parallel to the exterior surface 15 of the outer casing 12 at the distal end 11 of the outer casing 12, the third angle α3 may be equal to or substantially equal to an angle at which the injection sit engaging surface 26 of the tilting assist member 24 intersects the exterior surface 15 of the outer casing 12 at the distal end 11 of the outer casing 12.

A method of using the injection device 10 to subcutaneously deliver a dose of the drug 52 to the patient in accordance with one embodiment of the present disclosure will now be described with reference to FIGS. 3A-3G. As a preliminary step, the user, who in some instances may be the patient, may remove the cap 14 from the distal end 11 of the outer casing 12 to expose the guard member 62. In some embodiments, removal of the cap 14 may also result in removal of the shield 71, which may be frictionally or mechanically gripped by an interior structure of the cap 14. Then, as shown in FIG. 3A, the user may move the guard member 62 into contact with the patient's skin 90 at the injection site 92. At this phase of the process, the longitudinal axis Al of the subcutaneous delivery member 42 may be oriented perpendicular to or substantially perpendicular to the peripheral portion 93b of the injection site 92 and the central portion 93a of the injection site 92. This may be referred to as the first orientation of the subcutaneous delivery member 42 relative to the injection site 92. Next, while grasping the proximal end 13 of the outer casing 12 in one hand, the user may push the injection device 10 in the distal direction toward the injection site. This motion may cause the guard member 62 to retract into the outer casing 12, which, in turn, causes the distal end 49 of the subcutaneous delivery member 42, whose position is stationary relative to the outer casing 12, to pierce the patient's skin 90 as illustrated in FIG. 3B. During the insertion process and at least immediately thereafter, the first orientation of the subcutaneous delivery member 42 relative the injection site 92 may be maintained by the user.

Subsequently, after considering (e.g., reading) the instructional marker 20, the user may tilt the injection device 10 in the tilting direction designated by the instructional marker 20 (see FIG. 3C). In the illustrated embodiment, tilting the injection device 10 involves tilting the subcutaneous delivery member 42 in a direction away from the second lateral side 61 of the subcutaneous delivery member 42 (i.e., in a direction towards the far right-hand side of the sheet including FIG. 3C) such that the opening 53 is directed more so in the distal direction (i.e., away from the surface of the patient's skin 90 at the injection site 92). In some embodiments, this tilting motion may involve rotating the injection device 10 about an imaginary point defined by the distal end 49 of the subcutaneous delivery member 42 or by the distal end the guard member 62. By rotating the injection device 10 about an imaginary point defined by the distal end 49 of the subcutaneous delivery member 42, it may be possible to maintain the depth at which the distal end 49 of the subcutaneous delivery member 42 has been inserted into the patient's tissue. Furthermore, by rotating the injection device 10 about an imaginary point defined by the distal end 49 of the subcutaneous delivery member 42, it may be possible to maintain a constant or substantially constant angle or orientation of the longitudinal axis A1 of the subcutaneous delivery member 42 relative to a central portion 93b of the injection site 92. For example, the longitudinal axis A1 of the subcutaneous delivery member 42 may be arranged at a perpendicular or substantially perpendicular angle relative to the central portion 93b of the injection site 92 both before tilting (see FIG. 3B) and after tilting (see FIG. 3C). In some embodiments, rotating the injection device 10 about an imaginary point defined by the distal end 49 of the subcutaneous delivery member 42 may involve the patient exerting a downward force against the central portion 93b at the injection site 92 with the guard member 92 and/or the distal end 11 of the outer casing 12, thereby forming a temporary depression in the patient's skin 90 at the injection site 92, as depicted in FIG. 3C.

In some embodiments, the user may tilt the injection device 10 and hold the injection device 10 against the patient's skin 90 with a single hand. In other embodiments, the user may grip the proximal end 13 of the outer casing 12 with one hand to hold the injection device 10 against the patient's skin 90 while simultaneously pushing down on the finger grip surface 28 of the tilting assist member 24 with the user's other hand to tilt the injection device 10. In this way, the tilting assist member 24 may provide the user with leverage, allowing the user to steadily tilt the injection device 10 in a controlled manner. The user may continue tilting the injection device 10 until the injection site engaging surface 26 of the tilting assist member 24 contacts and/or is flush with the patient's skin 90 at the peripheral portion 93a of the injection site 92, as depicted in FIG. 3C. When the injection site engaging surface 26 of the tilting assist member 24 contacts and/or is flush with the patient's skin 90 at the peripheral portion 93a of the injection site 92, the longitudinal axis A1 of the subcutaneous delivery member 42 may be oriented relative to the patient's tissue in a manner that is optimal for reducing injection resistance. The patient or user will know he or she has reached this optimal orientation by virtue of the injection site engaging surface 26 of the tilting assist member 24 contacting and/or being flush with the patient's skin 90 at the peripheral portion 93a of the injection site 92. The amount of tilting that is needed for the injection site engaging surface 26 to contact and/or flushly engage the patient's skin 90 depends on the third angle α3. In some embodiments, once tilting is complete, the longitudinal axis A1 of the subcutaneous delivery member 42 may be arranged at an injection angle relative to the peripheral portion 93a of the injection site 92 which is equal to or substantially equal to the third angle α3. Accordingly, the injection angle may be equal to any of the values or ranges mentioned above for the third angle α3, or any other suitable angle for reducing injection resistance. In the configuration depicted in FIG. 3C, the sum of the first angle α1, the second angle α, and the injection angle may be equal to or substantially equal to 180 degrees. Once tilted, the subcutaneous delivery member 42 be referred to as having a second orientation relative to the injection site 92. In the second orientation, the longitudinal axis A1 of the subcutaneous delivery member 42 may, in some embodiments, be arranged perpendicular or substantially perpendicular to the central portion 93b of the injection site 92 but non-perpendicular to the peripheral portion 93a of the injection site 92, as illustrated in FIG. 3C.

Next, as illustrated in FIG. 3D, the user may depress the button member 16 to overcome the biasing force of the trigger biasing member 83. As a result, the detent member 80 may be disengaged from the plunger 54, which, in turn, allows the energy source 66 to propel the plunger 54 into the drug storage container 30 and initially impact the stopper 48. In one continuous motion, the plunger 54 may subsequently drive the stopper 48 in the distal direction through the interior chamber 50 to expel the drug 52 into the subcutaneous delivery member 42 and out through the opening 53 subcutaneously to the patient. Drug delivery may be completed when the plunger 54 reaches the end of its stroke as shown in FIG. 3E. It is noted that although the various contours formed in the patient's skin 90 at the injection site 92 by the injection device 10 are depicted as intersecting are relatively sharp corners in FIGS. 3C-3E, in at least some embodiments, the transitions between various contours formed in the patient's skin 90 at the injection site 92 may be more gradual and/or have at least some curvature to them.

Subsequently, as depicted in FIG. 3F, the user may tilt the injection device 10 back to first orientation shown in FIG. 3B, such that the longitudinal axis A1 of the subcutaneous delivery member 42 is once again oriented perpendicular to or substantially perpendicular to the patient's skin at the injection site 92. Finally, the user may lift the injection device 10 away from the patient to remove the subcutaneous delivery member 42 from the patient, as shown in FIG. 3G. As a result, the biasing member 64 may move the guard member 62 back to its extended position covering the distal end 49 of the subcutaneous delivery member 42. In some embodiments, the user may not tilt the injection device 10 back to the first orientation, and instead may remove the subcutaneous delivery member 42 from the patient while maintaining the second orientation (i.e., the tilted orientation) of the subcutaneous delivery member 42.

To ensure that the instructional marker 20 indicates the proper tilting direction, it may be important to install the subcutaneous delivery member 42 in a pre-defined position relative to the outer casing 12. Described below are methods of assembling (e.g., manufacturing) the injection device 10 that facilitate proper alignment of the subcutaneous delivery member 42 and the instructional marker 20.

As a preliminary step, the location of the opening 53 (or multiple openings) and/or the bevel 57 (or multiple bevels) at the distal end 49 of the subcutaneous delivery member 42 may be identified. In some embodiments, this step may involve identifying the circumferential position of one or more of these features about the longitudinal axis A1 of the subcutaneous delivery member 42. This may be done by visual inspection by a person and/or automatically with an imaging device. In some embodiments, including those where a needle shield 71 covers and thus hides the subcutaneous delivery member 42 from view, the imaging device may generate X-rays that pass through the needle shield 71 and the distal end 49 of the subcutaneous delivery member 42 and are subsequently received by a detector for analysis. In embodiments where the drug storage container 30 is a pre-filled syringe, the manufacturer of the pre-filled syringe may print or otherwise dispose a marker on an exterior surface of the body of the pre-filled syringe that is aligned with and/or indicates the circumferential position of the opening 53 (or multiple openings) and/or the bevel 57 (or multiple bevels).

Next, the instructional marker 20 may be disposed on the outer casing 12. However, this step may occur at any phase of the assembly process, including at the very end or the very beginning. The instructional marker 20 may be applied to the outer casing 12 in any suitable manner, including being printed directly on the exterior surface 15 of the outer casing 12 or being formed as a decal adhered to the exterior surface 15 of the outer casing 12. Furthermore, in some embodiments, the instructional marker 20 may be installed in the outer casing 12 as an electronic display, such as an LED display panel or individual lights, or as a fluid chamber housing a bubble.

The drug storage container 30 may then be disposed within the outer casing 12. Simultaneously or subsequently, the drug storage container 30 may be rotationally aligned with a target portion of the outer casing 12. In some embodiments, this step may involve rotationally aligning the opening 53 (or multiple openings) and/or the bevel 57 (or multiple bevels) and the target portion of the outer casing 12, based on the previously-identified circumferential position of the opening 53 (or multiple openings) and/or the bevel 57 (or multiple bevels) about the longitudinal axis A1 of the subcutaneous delivery member 42. This step may involve rotating the drug storage container 30 relative to the outer casing 12 after, or in some cases before, disposing the drug storage container 30 in the outer casing 12. Upon completion of this step, the relative positioning of the instructional marker 20, the target portion of the outer casing 12, and the opening 53 (or multiple openings) and/or the bevel 57 (or multiple bevels) may ensure that the instructional marker 20 indicates an optimal tilting direction for reducing injection resistance (i.e., a tilting direction that inhibits or prevents the patient's tissue from occluding the opening 53 (or multiple openings) in the distal end 49 of the subcutaneous delivery member 42 during drug delivery).

As an optional step following rotational alignment of the drug storage container 30 relative to the outer casing 12, the drug storage container 30 may be rotationally fixed relative to the outer casing 12 so that the drug storage container 30 is inhibited or preventing from rotating relative to the outer casing 12. As described above, the drug storage container 30 may be rotationally fixed relative to the outer casing 12 by the engagement structures 58, for example.

Furthermore, in embodiments where the drug storage container 30 is a pre-filled syringe having a shield (e.g., a rigid needle shield) covering and maintaining sterility of the subcutaneous delivery member 42, any one or combination of, or all of, the above-described assembly steps may be carried out in a non-sterile environment. In other embodiments, all of the above-described assembly steps may be carried out in a sterile environment.

From the foregoing, it can be seen that the present disclosure advantageously provides injection devices, as well as methods of using and assembling such devices, that reduce the injection resistance experienced during the subcutaneous delivery of a drug with the injection device. The mitigation of injection resistance may result in a quicker, more comfortable, and potentially safer injection for the patient. Furthermore, the reduced injection resistance may allow for the injection device to be configured with a less powerful injection drive mechanism. This in turn may reduce the possibility of damage to the drug storage container and/or the drug, reduce noise and/or vibrations caused by operation of the drive mechanism, facilitate smaller designs, and/or reduce costs.

Drug Information

As mentioned above, the drug storage container may be filled with a drug. This drug may be any one or combination of the drugs listed below, with the caveat that the following list should neither be considered to be all inclusive nor limiting.

For example, the syringe may be filled with colony stimulating factors, such as granulocyte colony-stimulating factor (G-CSF). Such G-CSF agents include, but are not limited to, Neupogen® (filgrastim) and Neulasta® (pegfilgrastim). In various other embodiments, the syringe may be used with various pharmaceutical products, such as an erythropoiesis stimulating agent (ESA), which may be in a liquid or a lyophilized form. An ESA is any molecule that stimulates erythropoiesis, such as Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin zeta, epoetin theta, and epoetin delta, as well as the molecules or variants or analogs thereof as disclosed in the following patents or patent applications, each of which is herein incorporated by reference in its entirety: U.S. Pat. Nos. 4,703,008; 5,441,868; 5,547,933; 5,618,698; 5,621,080; 5,756,349; 5,767,078; 5,773,569; 5,955,422; 5,986,047; 6,583,272; 7,084,245; and 7,271,689; and PCT Publication Nos. WO 91/05867; WO 95/05465; WO 96/40772; WO 00/24893; WO 01/81405; and WO 2007/136752.

An ESA can be an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, epoetin alfa, epoetin beta, epoetin delta, epoetin omega, epoetin iota, epoetin zeta, and analogs thereof, pegylated erythropoietin, carbamylated erythropoietin, mimetic peptides (including EMP1/hematide), and mimetic antibodies. Exemplary erythropoiesis stimulating proteins include erythropoietin, darbepoetin, erythropoietin agonist variants, and peptides or antibodies that bind and activate erythropoietin receptor (and include compounds reported in U.S. Publication Nos. 2003/0215444 and 2006/0040858, the disclosures of each of which is incorporated herein by reference in its entirety) as well as erythropoietin molecules or variants or analogs thereof as disclosed in the following patents or patent applications, which are each herein incorporated by reference in its entirety: U.S. Pat. Nos. 4,703,008; 5,441,868; 5,547,933; 5,618,698; 5,621,080; 5,756,349; 5,767,078; 5,773,569; 5,955,422; 5,830,851; 5,856,298; 5,986,047; 6,030,086; 6,310,078; 6,391,633; 6,583,272; 6,586,398; 6,900,292; 6,750,369; 7,030,226; 7,084,245; and 7,217,689; U.S. Publication Nos. 2002/0155998; 2003/0077753; 2003/0082749; 2003/0143202; 2004/0009902; 2004/0071694; 2004/0091961; 2004/0143857; 2004/0157293; 2004/0175379; 2004/0175824; 2004/0229318; 2004/0248815; 2004/0266690; 2005/0019914; 2005/0026834; 2005/0096461; 2005/0107297; 2005/0107591; 2005/0124045; 2005/0124564; 2005/0137329; 2005/0142642; 2005/0143292; 2005/0153879; 2005/0158822; 2005/0158832; 2005/0170457; 2005/0181359; 2005/0181482; 2005/0192211; 2005/0202538; 2005/0227289; 2005/0244409; 2006/0088906; and 2006/0111279; and PCT Publication Nos. WO 91/05867; WO 95/05465; WO 99/66054; WO 00/24893; WO 01/81405; WO 00/61637; WO 01/36489; WO 02/014356; WO 02/19963; WO 02/20034; WO 02/49673; WO 02/085940; WO 03/029291; WO 2003/055526; WO 2003/084477; WO 2003/094858; WO 2004/002417; WO 2004/002424; WO 2004/009627; WO 2004/024761; WO 2004/033651; WO 2004/035603; WO 2004/043382; WO 2004/101600; WO 2004/101606; WO 2004/101611; WO 2004/106373; WO 2004/018667; WO 2005/001025; WO 2005/001136; WO 2005/021579; WO 2005/025606; WO 2005/032460; WO 2005/051327; WO 2005/063808; WO 2005/063809; WO 2005/070451; WO 2005/081687; WO 2005/084711; WO 2005/103076; WO 2005/100403; WO 2005/092369; WO 2006/50959; WO 2006/02646; and WO 2006/29094.

Examples of other pharmaceutical products for use with the device may include, but are not limited to, antibodies such as Vectibix® (panitumumab), Xgeva™ (denosumab) and Prolia™ (denosamab); other biological agents such as Enbrel® (etanercept, TNF-receptor/Fc fusion protein, TNF blocker), Neulasta® (pegfilgrastim, pegylated filgastrim, pegylated G-CSF, pegylated hu-Met-G-CSF), Neupogen® (filgrastim, G-CSF, hu-MetG-CSF), and Nplate® (romiplostim); small molecule drugs such as Sensipar® (cinacalcet). The device may also be used with a therapeutic antibody, a polypeptide, a protein or other chemical, such as an iron, for example, ferumoxytol, iron dextrans, ferric glyconate, and iron sucrose. The pharmaceutical product may be in liquid form, or reconstituted from lyophilized form.

Among particular illustrative proteins are the specific proteins set forth below, including fusions, fragments, analogs, variants or derivatives thereof:

OPGL specific antibodies, peptibodies, and related proteins, and the like (also referred to as RANKL specific antibodies, peptibodies and the like), including fully humanized and human OPGL specific antibodies, particularly fully humanized monoclonal antibodies, including but not limited to the antibodies described in PCT Publication No. WO 03/002713, which is incorporated herein in its entirety as to OPGL specific antibodies and antibody related proteins, particularly those having the sequences set forth therein, particularly, but not limited to, those denoted therein: 9H7; 18B2; 2D8; 2E11; 16E1; and 22B3, including the OPGL specific antibodies having either the light chain of SEQ ID NO:2 as set forth therein in FIG. 2 and/or the heavy chain of SEQ ID NO:4, as set forth therein in FIG. 4, each of which is individually and specifically incorporated by reference herein in its entirety fully as disclosed in the foregoing publication;

Myostatin binding proteins, peptibodies, and related proteins, and the like, including myostatin specific peptibodies, particularly those described in U.S. Publication No. 2004/0181033 and PCT Publication No. WO 2004/058988, which are incorporated by reference herein in their entirety particularly in parts pertinent to myostatin specific peptibodies, including but not limited to peptibodies of the mTN8-19 family, including those of SEQ ID NOS:305-351, including TN8-19-1 through TN8-19-40, TN8-19 con1 and TN8-19 con2; peptibodies of the mL2 family of SEQ ID NOS:357-383; the mL15 family of SEQ ID NOS:384-409; the mL17 family of SEQ ID NOS:410-438; the mL20 family of SEQ ID NOS:439-446; the mL21 family of SEQ ID NOS:447-452; the mL24 family of SEQ ID NOS:453-454; and those of SEQ ID NOS:615-631, each of which is individually and specifically incorporated by reference herein in their entirety fully as disclosed in the foregoing publication;

IL-4 receptor specific antibodies, peptibodies, and related proteins, and the like, particularly those that inhibit activities mediated by binding of IL-4 and/or IL-13 to the receptor, including those described in PCT Publication No. WO 2005/047331 or PCT Application No. PCT/US2004/37242 and in U.S. Publication No. 2005/112694, which are incorporated herein by reference in their entirety particularly in parts pertinent to IL-4 receptor specific antibodies, particularly such antibodies as are described therein, particularly, and without limitation, those designated therein: L1H1; L1H2; L1H3; L1H4; L1H5; L1H6; L1H7; L1H8; L1H9; L1H10; L1H11; L2H1; L2H2; L2H3; L2H4; L2H5; L2H6; L2H7; L2H8; L2H9; L2H10; L2H11; L2H12; L2H13; L2H14; L3H1; L4H1; L5H1; L6H1, each of which is individually and specifically incorporated by reference herein in its entirety fully as disclosed in the foregoing publication;

Interleukin 1-receptor 1 (“IL1-R1”) specific antibodies, peptibodies, and related proteins, and the like, including but not limited to those described in U.S. Publication No. 2004/097712, which is incorporated herein by reference in its entirety in parts pertinent to IL1-R1 specific binding proteins, monoclonal antibodies in particular, especially, without limitation, those designated therein: 15CA, 26F5, 27F2, 24E12, and 10H7, each of which is individually and specifically incorporated by reference herein in its entirety fully as disclosed in the aforementioned publication;

Ang2 specific antibodies, peptibodies, and related proteins, and the like, including but not limited to those described in PCT Publication No. WO 03/057134 and U.S. Publication No. 2003/0229023, each of which is incorporated herein by reference in its entirety particularly in parts pertinent to Ang2 specific antibodies and peptibodies and the like, especially those of sequences described therein and including but not limited to: L1(N); L1(N) WT; L1(N) 1K WT; 2xL1(N); 2xL1(N) WT; Con4 (N), Con4 (N) 1K WT, 2xCon4 (N) 1K; L1C; L1C 1K; 2xL1C; Con4C; Con4C 1K; 2xCon4C 1K; Con4-L1 (N); Con4-L1C; TN-12-9 (N); C17 (N); TN8-8(N); TN8-14 (N); Con 1 (N), also including anti-Ang 2 antibodies and formulations such as those described in PCT Publication No. WO 2003/030833 which is incorporated herein by reference in its entirety as to the same, particularly Ab526; Ab528; Ab531; Ab533; Ab535; Ab536; Ab537; Ab540; Ab543; Ab544; Ab545; Ab546; A551; Ab553; Ab555; Ab558; Ab559; Ab565; AbF1AbFD; AbFE; AbFJ; AbFK; AbG1D4; AbGC1E8; AbH1C12; AbIA1; AbIF; AbIK, AbIP; and AbIP, in their various permutations as described therein, each of which is individually and specifically incorporated by reference herein in its entirety fully as disclosed in the foregoing publication;

NGF specific antibodies, peptibodies, and related proteins, and the like including, in particular, but not limited to those described in U.S. Publication No. 2005/0074821 and U.S. Pat. No. 6,919,426, which are incorporated herein by reference in their entirety particularly as to NGF-specific antibodies and related proteins in this regard, including in particular, but not limited to, the NGF-specific antibodies therein designated 4D4, 4G6, 6H9, 7H2, 14D10 and 14D11, each of which is individually and specifically incorporated by reference herein in its entirety fully as disclosed in the foregoing publication;

CD22 specific antibodies, peptibodies, and related proteins, and the like, such as those described in U.S. Pat. No. 5,789,554, which is incorporated herein by reference in its entirety as to CD22 specific antibodies and related proteins, particularly human CD22 specific antibodies, such as but not limited to humanized and fully human antibodies, including but not limited to humanized and fully human monoclonal antibodies, particularly including but not limited to human CD22 specific IgG antibodies, such as, for instance, a dimer of a human-mouse monoclonal hLL2 gamma-chain disulfide linked to a human-mouse monoclonal hLL2 kappa-chain, including, but limited to, for example, the human CD22 specific fully humanized antibody in Epratuzumab, CAS registry number 501423-23-0;

IGF-1 receptor specific antibodies, peptibodies, and related proteins, and the like, such as those described in PCT Publication No. WO 06/069202, which is incorporated herein by reference in its entirety as to IGF-1 receptor specific antibodies and related proteins, including but not limited to the IGF-1 specific antibodies therein designated L1H1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47H47, L48H48, L49H49, L50H50, L51H51, L52H52, and IGF-1R-binding fragments and derivatives thereof, each of which is individually and specifically incorporated by reference herein in its entirety fully as disclosed in the foregoing publication;

Also among non-limiting examples of anti-IGF-1R antibodies for use in the methods and compositions of the present disclosure are each and all of those described in:

(i) U.S. Publication No. 2006/0040358 (published Feb. 23, 2006), 2005/0008642 (published Jan. 13, 2005), 2004/0228859 (published Nov. 18, 2004), including but not limited to, for instance, antibody 1A (DSMZ Deposit No. DSM ACC 2586), antibody 8 (DSMZ Deposit No. DSM ACC 2589), antibody 23 (DSMZ Deposit No. DSM ACC 2588) and antibody 18 as described therein;

(ii) PCT Publication No. WO 06/138729 (published Dec. 28, 2006) and WO 05/016970 (published Feb. 24, 2005), and Lu et al. (2004), J. Biol. Chem. 279:2856-2865, including but not limited to antibodies 2F8, A12, and IMC-A12 as described therein;

(iii) PCT Publication No. WO 07/012614 (published Feb. 1, 2007), WO 07/000328 (published Jan. 4, 2007), WO 06/013472 (published Feb. 9, 2006), WO 05/058967 (published Jun. 30, 2005), and WO 03/059951 (published Jul. 24, 2003);

(iv) U.S. Publication No. 2005/0084906 (published Apr. 21, 2005), including but not limited to antibody 7C10, chimaeric antibody C7C10, antibody h7C10, antibody 7H2M, chimaeric antibody *7C10, antibody GM 607, humanized antibody 7C10 version 1, humanized antibody 7C10 version 2, humanized antibody 7C10 version 3, and antibody 7H2HM, as described therein;

(v) U.S. Publication Nos. 2005/0249728 (published Nov. 10, 2005), 2005/0186203 (published Aug. 25, 2005), 2004/0265307 (published Dec. 30, 2004), and 2003/0235582 (published Dec. 25, 2003) and Maloney et al. (2003), Cancer Res. 63:5073-5083, including but not limited to antibody EM164, resurfaced EM164, humanized EM164, huEM164 v1.0, huEM164 v1.1, huEM164 v1.2, and huEM164 v1.3 as described therein;

(vi) U.S. Pat. No. 7,037,498 (issued May 2, 2006), U.S. Publication Nos. 2005/0244408 (published Nov. 30, 2005) and 2004/0086503 (published May 6, 2004), and Cohen, et al. (2005), Clinical Cancer Res. 11:2063-2073, e.g., antibody CP-751,871, including but not limited to each of the antibodies produced by the hybridomas having the ATCC accession numbers PTA-2792, PTA-2788, PTA-2790, PTA-2791, PTA-2789, PTA-2793, and antibodies 2.12.1, 2.13.2, 2.14.3, 3.1.1, 4.9.2, and 4.17.3, as described therein;

(vii) U.S. Publication Nos. 2005/0136063 (published Jun. 23, 2005) and 2004/0018191 (published Jan. 29, 2004), including but not limited to antibody 19D12 and an antibody comprising a heavy chain encoded by a polynucleotide in plasmid 15H12/19D12 HCA (γ4), deposited at the ATCC under number PTA-5214, and a light chain encoded by a polynucleotide in plasmid 15H12/19D12 LCF (κ), deposited at the ATCC under number PTA-5220, as described therein; and

(viii) U.S. Publication No. 2004/0202655 (published Oct. 14, 2004), including but not limited to antibodies PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5, as described therein; each and all of which are herein incorporated by reference in their entireties, particularly as to the aforementioned antibodies, peptibodies, and related proteins and the like that target IGF-1 receptors;

B-7 related protein 1 specific antibodies, peptibodies, related proteins and the like (“B7RP-1,” also is referred to in the literature as B7H2, ICOSL, B7h, and CD275), particularly B7RP-specific fully human monoclonal IgG2 antibodies, particularly fully human IgG2 monoclonal antibody that binds an epitope in the first immunoglobulin-like domain of B7RP-1, especially those that inhibit the interaction of B7RP-1 with its natural receptor, ICOS, on activated T cells in particular, especially, in all of the foregoing regards, those disclosed in U.S. Publication No. 2008/0166352 and PCT Publication No. WO 07/011941, which are incorporated herein by reference in their entireties as to such antibodies and related proteins, including but not limited to antibodies designated therein as follow: 16H (having light chain variable and heavy chain variable sequences SEQ ID NO:1 and SEQ ID NO:7 respectively therein); 5D (having light chain variable and heavy chain variable sequences SEQ ID NO:2 and SEQ ID NO:9 respectively therein); 2H (having light chain variable and heavy chain variable sequences SEQ ID NO:3 and SEQ ID NO:10 respectively therein); 43H (having light chain variable and heavy chain variable sequences SEQ ID NO:6 and SEQ ID NO:14 respectively therein); 41H (having light chain variable and heavy chain variable sequences SEQ ID NO:5 and SEQ ID NO:13 respectively therein); and 15H (having light chain variable and heavy chain variable sequences SEQ ID NO:4 and SEQ ID NO:12 respectively therein), each of which is individually and specifically incorporated by reference herein in its entirety fully as disclosed in the foregoing publication;

IL-15 specific antibodies, peptibodies, and related proteins, and the like, such as, in particular, humanized monoclonal antibodies, particularly antibodies such as those disclosed in U.S. Publication Nos. 2003/0138421; 2003/023586; and 2004/0071702; and U.S. Pat. No. 7,153,507, each of which is incorporated herein by reference in its entirety as to IL-15 specific antibodies and related proteins, including peptibodies, including particularly, for instance, but not limited to, HuMax IL-15 antibodies and related proteins, such as, for instance, 14687;

IFN gamma specific antibodies, peptibodies, and related proteins and the like, especially human IFN gamma specific antibodies, particularly fully human anti-IFN gamma antibodies, such as, for instance, those described in U.S. Publication No. 2005/0004353, which is incorporated herein by reference in its entirety as to IFN gamma specific antibodies, particularly, for example, the antibodies therein designated 1118; 1118*; 1119; 1121; and 1121*. The entire sequences of the heavy and light chains of each of these antibodies, as well as the sequences of their heavy and light chain variable regions and complementarity determining regions, are each individually and specifically incorporated by reference herein in its entirety fully as disclosed in the foregoing publication and in Thakur et al. (1999), Mol. Immunol. 36:1107-1115. In addition, description of the properties of these antibodies provided in the foregoing publication is also incorporated by reference herein in its entirety. Specific antibodies include those having the heavy chain of SEQ ID NO:17 and the light chain of SEQ ID NO:18; those having the heavy chain variable region of SEQ ID NO:6 and the light chain variable region of SEQ ID NO:8; those having the heavy chain of SEQ ID NO:19 and the light chain of SEQ ID NO:20; those having the heavy chain variable region of SEQ ID NO:10 and the light chain variable region of SEQ ID NO:12; those having the heavy chain of SEQ ID NO:32 and the light chain of SEQ ID NO:20; those having the heavy chain variable region of SEQ ID NO:30 and the light chain variable region of SEQ ID NO:12; those having the heavy chain sequence of SEQ ID NO:21 and the light chain sequence of SEQ ID NO:22; those having the heavy chain variable region of SEQ ID NO:14 and the light chain variable region of SEQ ID NO:16; those having the heavy chain of SEQ ID NO:21 and the light chain of SEQ ID NO:33; and those having the heavy chain variable region of SEQ ID NO:14 and the light chain variable region of SEQ ID NO:31, as disclosed in the foregoing publication. A specific antibody contemplated is antibody 1119 as disclosed in the foregoing U.S. publication and having a complete heavy chain of SEQ ID NO:17 as disclosed therein and having a complete light chain of SEQ ID NO:18 as disclosed therein;

TALL-1 specific antibodies, peptibodies, and the related proteins, and the like, and other TALL specific binding proteins, such as those described in U.S. Publication Nos. 2003/0195156 and 2006/0135431, each of which is incorporated herein by reference in its entirety as to TALL-1 binding proteins, particularly the molecules of Tables 4 and 5B, each of which is individually and specifically incorporated by reference herein in its entirety fully as disclosed in the foregoing publications;

Parathyroid hormone (“PTH”) specific antibodies, peptibodies, and related proteins, and the like, such as those described in U.S. Pat. No. 6,756,480, which is incorporated herein by reference in its entirety, particularly in parts pertinent to proteins that bind PTH;

Thrombopoietin receptor (“TPO-R”) specific antibodies, peptibodies, and related proteins, and the like, such as those described in U.S. Pat. No. 6,835,809, which is herein incorporated by reference in its entirety, particularly in parts pertinent to proteins that bind TPO-R;

Hepatocyte growth factor (“HGF”) specific antibodies, peptibodies, and related proteins, and the like, including those that target the HGF/SF:cMet axis (HGF/SF:c-Met), such as the fully human monoclonal antibodies that neutralize hepatocyte growth factor/scatter (HGF/SF) described in U.S. Publication No. 2005/0118643 and PCT Publication No. WO 2005/017107, huL2G7 described in U.S. Pat. No. 7,220,410 and OA-5d5 described in U.S. Pat. Nos. 5,686,292 and 6,468,529 and in PCT Publication No. WO 96/38557, each of which is incorporated herein by reference in its entirety, particularly in parts pertinent to proteins that bind HGF;

TRAIL-R2 specific antibodies, peptibodies, related proteins and the like, such as those described in U.S. Pat. No. 7,521,048, which is herein incorporated by reference in its entirety, particularly in parts pertinent to proteins that bind TRAIL-R2;

Activin A specific antibodies, peptibodies, related proteins, and the like, including but not limited to those described in U.S. Publication No. 2009/0234106, which is herein incorporated by reference in its entirety, particularly in parts pertinent to proteins that bind Activin A;

TGF-beta specific antibodies, peptibodies, related proteins, and the like, including but not limited to those described in U.S. Pat. No. 6,803,453 and U.S. Publication No. 2007/0110747, each of which is herein incorporated by reference in its entirety, particularly in parts pertinent to proteins that bind TGF-beta;

Amyloid-beta protein specific antibodies, peptibodies, related proteins, and the like, including but not limited to those described in PCT Publication No. WO 2006/081171, which is herein incorporated by reference in its entirety, particularly in parts pertinent to proteins that bind amyloid-beta proteins. One antibody contemplated is an antibody having a heavy chain variable region comprising SEQ ID NO:8 and a light chain variable region having SEQ ID NO:6 as disclosed in the foregoing publication;

c-Kit specific antibodies, peptibodies, related proteins, and the like, including but not limited to those described in U.S. Publication No. 2007/0253951, which is incorporated herein by reference in its entirety, particularly in parts pertinent to proteins that bind c-Kit and/or other stem cell factor receptors;

OX40L specific antibodies, peptibodies, related proteins, and the like, including but not limited to those described in U.S. Publication No. 2006/0002929, which is incorporated herein by reference in its entirety, particularly in parts pertinent to proteins that bind OX40L and/or other ligands of the OX40 receptor; and

Other exemplary proteins, including Activase® (alteplase, tPA); Aranesp® (darbepoetin alfa); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Humatrope® (somatropin, Human Growth Hormone); Humira® (adalimumab); insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Neulasta® (pegylated filgastrim, pegylated G-CSF, pegylated hu-Met-G-CSF); Neupogen® (filgrastim , G-CSF, hu-MetG-CSF); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP Ilb/Ilia receptor monoclonal antibody); Actemra® (anti-IL6 Receptor mAb); Avastin® (bevacizumab), HuMax-CD4 (zanolimumab); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 146B7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Vectibix® (panitumumab); Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Rα mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-1103); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); anti-LLY antibody; BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; anti-ZP3 mAb (HuMax-ZP3); NVS Antibody #1; and NVS Antibody #2.

Also included can be a sclerostin antibody, such as but not limited to romosozumab, blosozumab, or BPS 804 (Novartis). Further included can be therapeutics such as rilotumumab, bixalomer, trebananib, ganitumab, conatumumab, motesanib diphosphate, brodalumab, vidupiprant, panitumumab, denosumab, NPLATE, PROLIA, VECTIBIX or XGEVA. Additionally, included in the device can be a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab), as well as molecules, variants, analogs or derivatives thereof as disclosed in the following patents or patent applications, each of which is herein incorporated by reference in its entirety for all purposes: U.S. Pat. Nos. 8,030,547, 8,563,698, 8,829,165, 8,859,741, 8,871,913, 8,871,914, 8,883,983, 8,889,834, 8,981,064, 9,056,915, 8,168,762, 9,045,547, 8,030,457, 8,030,457, 8,829,165, 8,981,064, 8,030,457, U.S. Publication No. 2013/0064825, U.S. Patent Application Publication No. 2012/0093818, U.S. Patent Application Publication No. 2013/0079502, U.S. Patent Application Publication No. 2014/0357850, U.S. Patent Application Publication No. 2011/0027287, U.S. Patent Application Publication No. 2014/0357851, U.S. Patent Application Publication No. 2014/0357854, U.S. Patent Application Publication No. 2015/0031870, U.S. Patent Application Publication No. 2013/0085265, U.S. Patent Application Publication No. 2013/0079501, U.S. Patent Application Publication No. 2012/0213797, U.S. Patent Application Publication No. 2012/0251544, U.S. Patent Application Publication No. 2013/0072665, U.S. Patent Application Publication No. 2013/0058944, U.S. Patent Application Publication No. 2013/0052201, U.S. Patent Application Publication No. 2012/0027765, U.S. Patent Application Publication No. 2015/0087819, U.S. Patent Application Publication No. 2011/0117011, U.S. Patent Application Publication No. 2015/0004174, U.S. Provisional Patent Application No. 60/957,668, U.S. Provisional Patent Application No. 61/008,965, U.S. Provisional Patent Application No. 61/010,630, U.S. Provisional Patent Application No. 61/086,133, U.S. Provisional Patent Application No. 61/125,304, U.S. Provisional Patent Application No. 61/798,970, U.S. Provisional Patent Application No. 61/841,039, U.S. Provisional Patent Application No. 62/002,623, U.S. Provisional Patent Application No. 62/024,399, U.S. Provisional Patent Application No. 62/019,729, U.S. Provisional Patent Application No. 62/067,637, U.S. patent application Ser. No. 14/777,371, International Patent Application No. PCT/US2013/048714, International Patent Application No. PCT/US2015/040211, International Patent Application No. PCT/US2015/056972, International Patent Application Publication No. WO/2008/057457, International Patent Application Publication No. WO/2008/057458, International Patent Application Publication No. WO/2008/057459, International Patent Application Publication No. WO/2008/063382, International Patent Application Publication No. WO/2008/133647, International Patent Application Publication No. WO/2009/100297, International Patent Application Publication No. WO/2009/100318, International Patent Application Publication No. WO/2011/037791, International Patent Application Publication No. WO/2011/053759, International Patent Application Publication No. WO/2011/053783, International Patent Application Publication No. WO/2008/125623, International Patent Application Publication No. WO/2011/072263, International Patent Application Publication No. WO/2009/055783, International Patent Application Publication No. WO/2012/0544438, International Patent Application Publication No. WO/2010/029513, International Patent Application Publication No. WO/2011/111007, International Patent Application Publication No. WO/2010/077854, International Patent Application Publication No. WO/2012/088313, International Patent Application Publication No. WO/2012/101251, International Patent Application Publication No. WO/2012/101252, International Patent Application Publication No. WO/2012/101253, International Patent Application Publication No. WO/2012/109530, and International Patent Application Publication No. WO/2001/031007, International Patent Application Publication No. WO/2009/026558, International Patent Application Publication No. WO/2009/131740, International Patent Application Publication No. WO/2013/166448, and International Patent Application Publication No. WO/2014/150983.

Also included can be talimogene laherparepvec or another oncolytic HSV for the treatment of melanoma or other cancers. Examples of oncolytic HSV include, but are not limited to talimogene laherparepvec (U.S. Pat. Nos. 7,223,593 and 7,537,924); OncoVEXGALV/CD (U.S. Pat. No. 7,981,669); OrienX010 (Lei et al. (2013), World J. Gastroenterol., 19:5138-5143); G207, 1716; NV1020; NV12023; NV1034 and NV1042 (Vargehes et al. (2002), Cancer Gene Ther., 9(12):967-978).

Also included are TIMPs. TIMPs are endogenous tissue inhibitors of metalloproteinases (TIMPs) and are important in many natural processes. TIMP-3 is expressed by various cells or and is present in the extracellular matrix; it inhibits all the major cartilage-degrading metalloproteases, and may play a role in role in many degradative diseases of connective tissue, including rheumatoid arthritis and osteoarthritis, as well as in cancer and cardiovascular conditions. The amino acid sequence of TIMP-3, and the nucleic acid sequence of a DNA that encodes TIMP-3, are disclosed in U.S. Pat. No. 6,562,596, issued May 13, 2003, the disclosure of which is incorporated by reference herein. Description of TIMP mutations can be found in U.S. Publication No. 2014/0274874 and PCT Publication No. WO 2014/152012.

Also included are antagonistic antibodies for human calcitonin gene-related peptide (CGRP) receptor and bispecific antibody molecule that target the CGRP receptor and other headache targets. Further information concerning these molecules can be found in PCT Application No. WO 2010/075238.

Additionally, a bispecific T cell engager antibody (BiTe), e.g. Blinotumomab can be used in the device. Alternatively, included can be an APJ large molecule agonist e.g., apelin or analogues thereof in the device. Information relating to such molecules can be found in PCT Publication No. WO 2014/099984.

In certain embodiments, the drug comprises a therapeutically effective amount of an anti-thymic stromal lymphopoietin (TSLP) or TSLP receptor antibody. Examples of anti-TSLP antibodies that may be used in such embodiments include, but are not limited to, those described in U.S. Pat. Nos. 7,982,016, and 8,232,372, and U.S. Publication No. 2009/0186022. Examples of anti-TSLP receptor antibodies include, but are not limited to, those described in U.S. Pat. No. 8,101,182. In particularly preferred embodiments, the drug comprises a therapeutically effective amount of the anti-TSLP antibody designated as A5 within U.S. Pat. No. 7,982,016.

While the present disclosure has been described in connection with various embodiments, it will be understood that the present disclosure is capable of further modifications. The present disclosure is intended to cover any variations, uses, or adaptations of the disclosed subject matter following, in general, the principles of the present disclosure, and including such departures from the present disclosure as, within the known and customary practice within the art to which the present disclosure pertains.

It is noted that the construction and arrangement of the injection device as shown in the various exemplary embodiments is illustrative only. Although only a few embodiments of the subject matter at issue have been described in detail in the present disclosure, those skilled in the art who review the present disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter disclosed herein. For example, elements shown as integrally formed may be constructed of multiple parts or elements, and vice versa. Also, the position of elements may be reversed or otherwise varied, and the nature or number of discrete elements or positions may be altered or varied. Accordingly, all such modifications are intended to be included within the scope of the present disclosure as defined in the appended claims. Furthermore, the order or sequence of any process or method steps may be varied or re-sequenced according to alternative embodiments. Other substitutions, modifications, changes and omissions may be made in the design, operating conditions and arrangement of the various exemplary embodiments without departing from the scope of the present disclosure.

1. An injection device for drug delivery, the injection device comprising:
an outer casing;
a container disposed in the outer casing and including
an interior chamber for storing a drug,
a subcutaneous delivery member, and
a stopper movably disposed in the interior chamber;
an injection drive mechanism configured to move the stopper to expel the drug through an opening in a distal end of the subcutaneous delivery member upon activation; and
an instructional marker disposed on an exterior surface of the outer casing, and indicating a tilting direction for tilting the injection device when the subcutaneous delivery member is inserted into a patient so as to inhibit tissue from occluding the opening in the distal end of the subcutaneous delivery member.

2. The injection device of claim 1, the opening being defined by a bevel formed in the distal end of the subcutaneous delivery member.

3. The injection device of claim 2, wherein the instructional marker indicates tilting the subcutaneous delivery member away from a lateral side of the subcutaneous delivery member including the bevel.

4. The injection device of claim 1, the instructional marker being printed on, adhered to, or installed adjacent the exterior surface of the outer casing.

5. The injection device of claim 1, the instructional marker being generated by an electronic display.

6. The injection device of claim 1, the instructional marker comprising a tilting assist member protruding radially outwardly from a distal end of the outer casing.

7. The injection device of claim 6, the tilting assist member including a finger grip surface arranged relative to a longitudinal axis of the subcutaneous delivery member at a first angle, and an injection site engaging surface arranged relative to the finger grip surface at second angle.

8. The injection device of claim 7, the injection site engaging surface of the tilting assist member being arranged relative to the longitudinal axis of the subcutaneous delivery member at a third angle, wherein a sum of the first angle, the second angle, and the third angle is substantially equal to 180 degrees.

9. The injection device of claim 1, wherein the subcutaneous delivery member is a rigid needle.

10. The injection device of claim 9, comprising a removable needle shield covering and maintaining sterility of the distal end of the subcutaneous delivery member prior to use.

11. A method of assembling an injection device for drug delivery, the method comprising:
disposing a container within an outer casing, the container including
an interior chamber for storing a drug,
a subcutaneous delivery member, and
a stopper movably disposed in the interior chamber to expel the drug through an opening defined by a bevel formed in a distal end of the delivery member;
rotationally aligning the bevel of the subcutaneous delivery member relative to a target portion of the outer casing; and
wherein the target portion is associated with a tilting direction for tilting the injection device when the subcutaneous delivery member is inserted into a patient so as to inhibit tissue from occluding the opening in the distal end of the subcutaneous delivery member.

12. The method of claim 11, comprising rotationally fixing the subcutaneous delivery member relative to the outer casing so as to inhibit rotation of the subcutaneous delivery member relative to outer casing after rotationally aligning the bevel relative to the target portion of the outer casing.

13. The method of claim 11, comprising disposing an injection drive mechanism within the outer casing, the injection drive mechanism being configured to move the stopper to expel the drug through an opening in a distal end of the subcutaneous delivery member upon activation.

14. The method of claim 11, wherein rotationally aligning the bevel of the subcutaneous delivery member relative to the target portion of the outer casing is performed after disposing the container within the outer casing.

15. The method of claim 11, wherein the interior chamber of the container is pre-filled with the drug.

16. The method of claim 11, wherein disposing the container within the outer casing is performed in a non-sterile environment.

17. The method of claim 11, comprising using an imaging device to identify a position of the bevel on the subcutaneous delivery member prior to rotationally aligning the bevel relative to the target portion of the outer casing.

18. The method of claim 17, wherein the imaging device includes an X-ray generator.

19. The method of claim 11, comprising marking an exterior surface of the outer casing with an instructional marker indicating the tilting direction for tilting the injection device when the subcutaneous delivery member is inserted into the patient so as to inhibit tissue from occluding the opening in the distal end of the subcutaneous delivery member.

20. The method of claim 19, wherein the instructional marker indicates tilting the subcutaneous delivery member away from a lateral side of the subcutaneous delivery member including the bevel.

21. A method of delivering a drug to a patient, the method comprising:
providing an injection device including
an outer casing,
a container disposed in the outer casing and including an interior chamber for storing the drug,
a subcutaneous delivery member,
a stopper movably disposed in the interior chamber, and
an injection drive mechanism configured to move the stopper to expel the drug through an opening in a distal end of the subcutaneous delivery member upon activation;
inserting the distal end of the subcutaneous delivery member into the patient with the subcutaneous delivery member having a first orientation relative to an injection site of the patient;
tilting the injection device in a first direction such that the subcutaneous delivery member has a second orientation relative to the injection site of the patient; and
activating the injection drive mechanism to subcutaneously deliver the drug to the patient while maintaining the subcutaneous delivery member in the second orientation relative to the injection site of the patient.

22. The method of claim 21, wherein tilting the injection device in the first direction such that the subcutaneous delivery member has the second orientation relative to the injection site of the patient comprises forming an angle between a longitudinal axis of the subcutaneous delivery member and a peripheral portion of the injection site of the patient that is less than or equal to 85 degrees.

23. The method of claim 21, comprising, upon completion of drug delivery, tilting the injection device in a second direction, opposite to the first direction, to return the subcutaneous delivery member to the first orientation relative to the injection site of the patient.

24. The method of claim 23, comprising removing the subcutaneous delivery member from the patient while maintaining the subcutaneous delivery member in the first orientation relative to the injection site of the patient.

25. The method of claim 21, wherein the outer casing of the injection device includes an instructional marker indicating the first direction for tilting the injection device.

26. The method of claim 21, wherein tilting the injection device in the first direction comprises engaging the injection site of the patient with a tilting assist member protruding radially outwardly from a distal end of the outer casing.

27. The method of claim 26, wherein tilting the injection device in the first direction such that the subcutaneous delivery member has the second orientation relative to the injection site of the patient comprises holding the outer casing in one hand and exerting a force against the tilting assist member in a direction toward the patient with another hand.
